(19) United States
(12) Patent Application Publication
(10) Pub. No.: US 2011/0105409 A1
(43) Pub. Date: May 5, 2011

(54) Anticancer Compounds

(75) Inventors: Raquel Rodríguez Acebes, Madrid (ES); Rogelio Fernández Rodríguez, Madrid (ES); José Fernando Reyes Benítez, Madrid (ES); Ma Jesús Martín López, Madrid (ES); Isabel Marco Martínez, Madrid (ES); Isabel Digón Juárez, Madrid (ES); Maria del Carmen Cuevas Marchante, Madrid (ES); Andrés Francesch Solloso, Madrid (ES)

(73) Assignee: PHARMA MAR, S.A., Madrid (ES)

(21) Appl. No.: 13/002,154

(22) PCT Filed: Jul. 16, 2009
(86) PCT No.: PCT/EP2009/059196

§ 371 Date: Dec. 30, 2010

(30) Foreign Application Priority Data
Jul. 17, 2008 (EP) 08380219.9

(51) Int. Cl. A61K 38/12; C07K 11/02; A61P 35/00

(52) U.S. Cl. 514/19.3; 530/321; 514/21.1

(57) ABSTRACT

Anticancer compounds of general formula I:

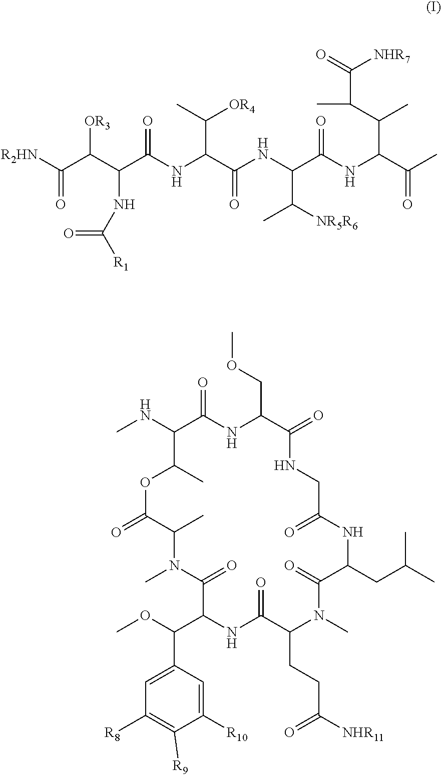

wherein R₁-R₁₁ take permitted meanings for use in the treatment of cancer.

### FIELD OF THE INVENTION

The present invention relates to new anticancer compounds, pharmaceutical compositions containing them and their use as anticancer agents.

### BACKGROUND OF THE INVENTION

Cyclic depsipeptides have emerged as a very important class of bioactive compounds from marine organisms. Several of these cyclic depsipeptides have been disclosed to have cytotoxic, antiviral and/or antifungal properties. Specifically, callipeltin A was disclosed to be isolated from the marine sponges Callipelta sp and Latrunculia sp (Zampella et al. J. Am. Chem. Soc. 1996, 118(26), 6202-6209; Zampella et al. Tetrahedron Letters, 2002, 43, 6163-6166), and callipeltin B from Callipelta sp (D'Auria et al. Tetrahedron, 1996, 52(28), 9589-9596).

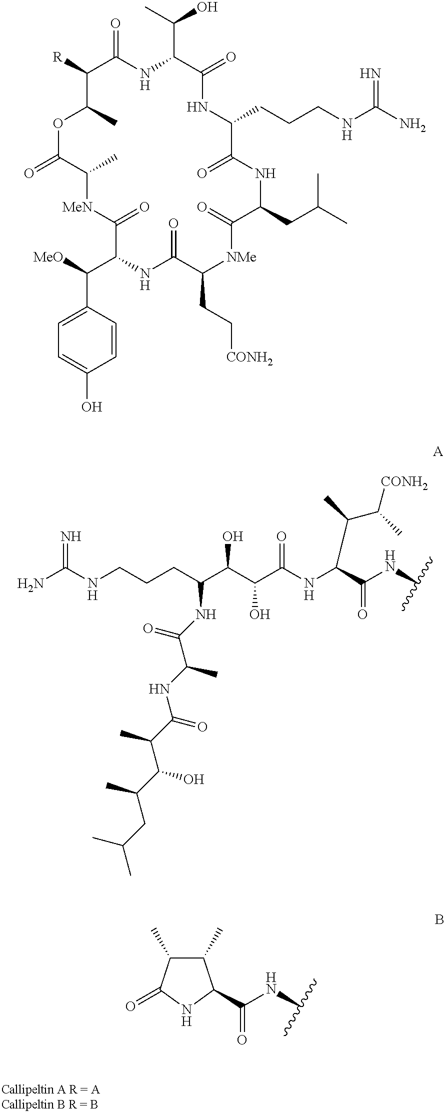

Zampella et al. reported that callipeltin A has antiviral and antifungal activities. Specifically, the antiviral activity of the compound was measured on CEM4 lymphocytic cell lines infected with HIV-1 (Lai strain). It was found, that the compound exhibited a CD₅₀ of 0.29 μg/mL and an ED₅₀ of 0.01 μg/mL giving a selectivity index (SI ratio CD₅₀/ED₅₀) of 29. In addition, the antifungal activity of callipeltin A was measured against Candida albicans, whose growth was inhibited at 100 μg/disc (6 mm) with 30 mm of inhibition (Zampella et al. J. Am. Chem. Soc. 1996, 118(26), 6202-6209).

D'Auria et al also reported that both callipeltin A and B are cytotoxic against various human carcinoma cells in vitro. Specifically, the cytotoxicity of both compounds was evaluated against NSCLC-N6 (human bronchopulmonary non-small-cell-lung-carcinoma), E39 (human renal carcinoma), P388 (murine leukaemia), and M96 (human melanoma) tumor cells, and it was found that callipeltin A exhibited a IC₅₀ ranging from <1.1 to >30 μg/mL and callipeltin B exhibited a IC₅₀ ranging from 1.3 to >30 μg/mL (D'Auria et al. Tetrahedron, 1996, 52(28), 9589-9596). Additionally, callipeltin A was found to be a selective and powerful inhibitor of the Na⁺/Ca²⁺ exchanger and a positive inotropic agent in guinea pig atria (Trevisi et al. Biochem. Biophys. Res. Commun. 2000, 279(1), 219-222).

In 1999, Ford et al. disclosed the isolation of four novel cyclic depsipeptides named papuamides A, B, C, and D from the sponges Theonella mirabilis and Theonella swinhoei. In addition, the synthesis of a diacetate derivative of papuamide A was disclosed (Ford et al. J. Am. Chem. Soc. 1999, 121(25), 5899-5909).

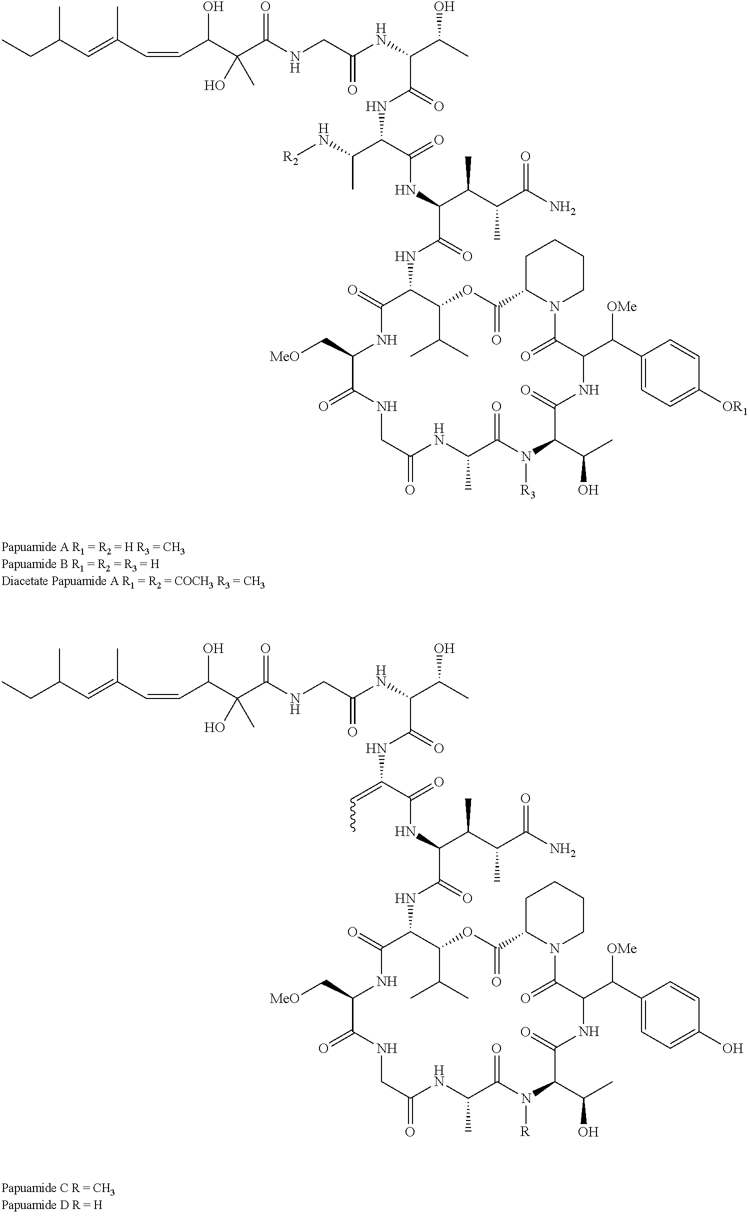

It was found that papuamides A and B inhibited the infection of human T-lymphoblastoid cells by HIV-1_{RF} in vitro with an EC₅₀ of approximately 4 ng/mL. In addition, papuamide A was found to be cytotoxic against a panel of human cancer cell lines with a mean IC₅₀ of 75 ng/mL.

An additional cyclic depsipeptide of the family of papuamides was disclosed by Ratnayake et al. Specifically, it was disclosed the isolation of theopapuamide from sponge Theonella swinhoei, which exhibited cytotoxic activity against CEM-TART (CEM T-cells expressing both HIV-1 tat and rev) and HCT-116 (human colon tumor) cell lines with EC50 values of 0.5 and 0.9 μM, respectively (Ratnayake et al. J. Nat. Prod. 2006, 69(11), 1582-1586).

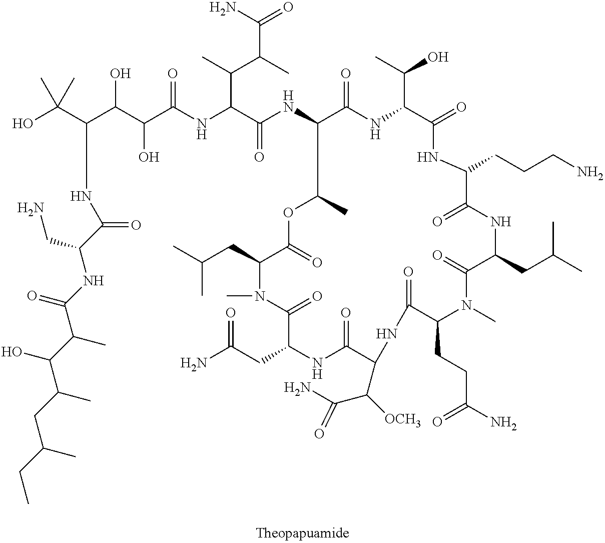

Finally, Oku et al. have also reported a new class of cyclic depsipeptides with antiviral activity. Specifically, they isolated neamphamide A from the sponge Neamphius huxleyi, which exhibited a potent cytoprotective activity against HIV-1 infection with an EC₅₀ of approximately 28 nM (Oku et al. J. Nat. Prod. 2004, 67(8), 1407-1411).

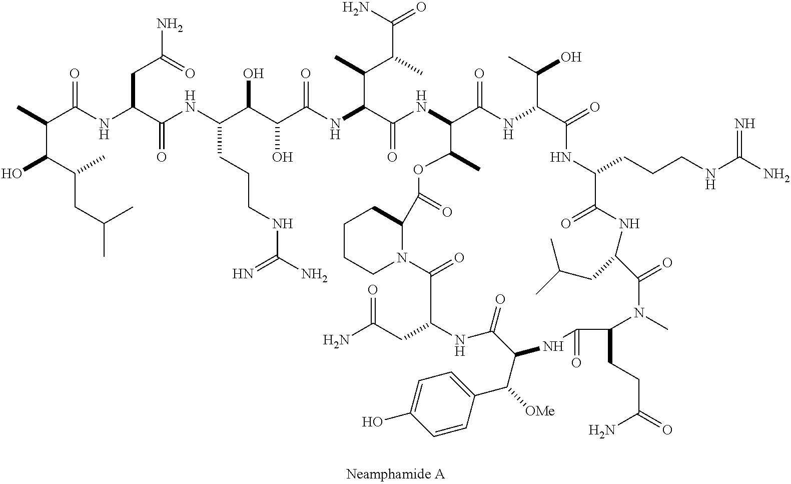

Since cancer is a leading cause of death in animals and humans, several efforts have been and are still being undertaken in order to obtain an anticancer therapy active and safe to be administered to patients suffering from a cancer. The problem to be solved by the present invention is to provide compounds that are useful in the treatment of cancer.

### SUMMARY OF THE INVENTION

In one aspect, the present invention is directed to compounds of general formula I or pharmaceutically acceptable salts, tautomers, prodrugs or stereoisomers thereof

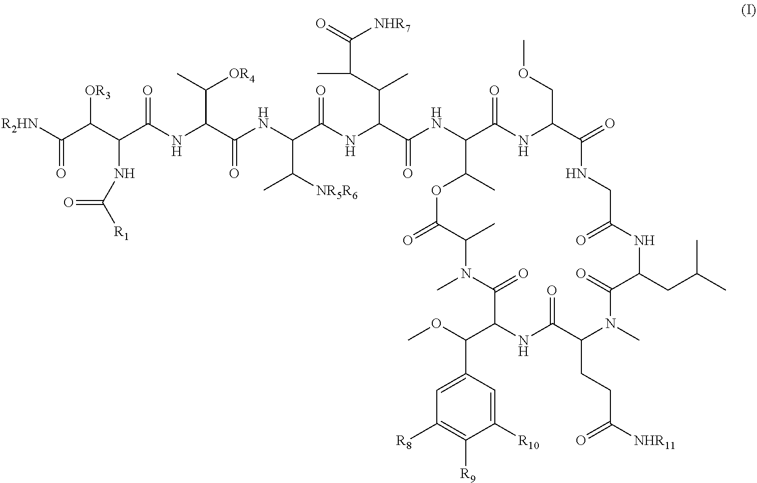

wherein
R₁ is selected from substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group;
each R₂, R₇, and R₁₁ is independently selected from hydrogen, CORₐ, COORₐ, CONRₐR_{b}, substituted or unsubstituted C₁-C₁₂ alkyl, substituted or unsubstituted C₂-C₁₂ alkenyl, and substituted or unsubstituted C₂-C₁₂ alkynyl;
each R₃ and R₄ is independently selected from hydrogen, CORₐ, COORₐ, CONRₐR_{b}, SO₂Rₐ, SO₃Rₐ, substituted or unsubstituted C₁-C₁₂ alkyl, substituted or unsubstituted C₂-C₁₂ alkenyl, and substituted or unsubstituted C₂-C₁₂ alkynyl;
each R₅ and R₆ is independently selected from hydrogen, CORₐ, COORₐ, substituted or unsubstituted C₁-C₁₂ alkyl, substituted or unsubstituted C₂-C₁₂ alkenyl, and substituted or unsubstituted C₂-C₁₂ alkynyl;
each R₈, R₉, and R₁₀ is independently selected from hydrogen, OR_{c}, CORₐ, COORₐ, CONRₐR_{b}, CN, NRₐR_{b}, halogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group;
R_{c} is selected from hydrogen, CORₐ, COORₐ, CONRₐR_{b}, SO₂Rₐ, SO₃Rₐ, substituted or unsubstituted C₁-C₁₂ alkyl, substituted or unsubstituted C₂-C₁₂ alkenyl, and substituted or unsubstituted C₂-C₁₂ alkynyl; and
each Rₐ and R_{b} is independently selected from hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group.

In another aspect, the present invention is directed to a compound of formula I, or a pharmaceutically acceptable salt, tautomer, prodrug or stereoisomer thereof, for use as a medicament, in particular as a medicament for treating cancer.

In a further aspect, the present invention is also directed to the use of a compound of formula I, or a pharmaceutically acceptable salt, tautomer, prodrug or stereoisomer thereof, in the treatment of cancer, or in the preparation of a medicament, preferably for the treatment of cancer. Other aspects of the invention are methods of treatment, and compounds for use in these methods. Therefore, the present invention further provides a method of treating a patient, notably a human, affected by cancer which comprises administering to said affected individual in need thereof a therapeutically effective amount of a compound as defined above.

In a yet further aspect, the present invention is also directed to a compound of formula I, or a pharmaceutically acceptable salt, tautomer, prodrug or stereoisomer thereof, for use as an anticancer agent.

In another aspect, the present invention is directed to pharmaceutical compositions comprising a compound of formula I, or a pharmaceutically acceptable salt, tautomer, prodrug or stereoisomer thereof, together with a pharmaceutically acceptable carrier or diluent.

The present invention also relates to the isolation of compounds of formula I from a sponge of the family Ancorinidae, genus Ecionemia, species Ecionemia acervus Bowerbank 1864, and the formation of derivatives from the isolated compounds.

### DETAILED DESCRIPTION OF THE INVENTION

The present invention relates to compounds of general formula I as defined above.

In these compounds the groups can be selected in accordance with the following guidance:

Alkyl groups may be branched or unbranched, and preferably have from 1 to about 12 carbon atoms. One more preferred class of alkyl groups has from 1 to about 6 carbon atoms. Even more preferred are alkyl groups having 1, 2, 3 or 4 carbon atoms. Methyl, ethyl, n-propyl, iso-propyl and butyl, including n-butyl, tert-butyl, sec-butyl and iso-butyl are particularly preferred alkyl groups in the compounds of the present invention. Another preferred class of alkyl groups has from 8 to about 12 carbon atoms; and even more preferably 9, 10 or 11 carbon atoms. As used herein, the term alkyl, unless otherwise stated, refers to both cyclic and noncyclic groups, although cyclic groups will comprise at least three carbon ring members.

Preferred alkenyl and alkynyl groups in the compounds of the present invention may be branched or unbranched, have one or more unsaturated linkages and from 2 to about 12 carbon atoms. One more preferred class of alkenyl and alkynyl groups has from 2 to about 6 carbon atoms. Even more preferred are alkenyl and alkynyl groups having 2, 3 or 4 carbon atoms. Another preferred class of alkenyl and alkynyl groups has from 8 to about 12 carbon atoms; and even more preferably 9, 10 or 11 carbon atoms. The terms alkenyl and alkynyl as used herein refer to both cyclic and noncyclic groups, although cyclic groups will comprise at least three carbon ring members.

Suitable aryl groups in the compounds of the present invention include single and multiple ring compounds, including multiple ring compounds that contain separate and/or fused aryl groups. Typical aryl groups contain from 1 to 3 separated or fused rings and from 6 to about 18 carbon ring atoms. Preferably aryl groups contain from 6 to about 10 carbon ring atoms. Specially preferred aryl groups include substituted or unsubstituted phenyl, substituted or unsubstituted naphthyl, substituted or unsubstituted biphenyl, substituted or unsubstituted phenanthryl and substituted or unsubstituted anthryl.

Suitable heterocyclic groups include heteroaromatic and heteroalicyclic groups containing from 1 to 3 separated and/or fused rings and from 5 to about 18 ring atoms. Preferably heteroaromatic and heteroalicyclic groups contain from 5 to about 10 ring atoms. Suitable heteroaromatic groups in the compounds of the present invention contain one, two or three heteroatoms selected from N, O or S atoms and include, e.g., coumarinyl including 8-coumarinyl, quinolyl including 8-quinolyl, isoquinolyl, pyridyl, pyrazinyl, pyrazolyl, pyrimidinyl, furyl, pyrrolyl, thienyl, thiazolyl, isothiazolyl, triazolyl, tetrazolyl, isoxazolyl, oxazolyl, imidazolyl, indolyl, isoindolyl, indazolyl, indolizinyl, phthalazinyl, pteridinyl, purinyl, oxadiazolyl, thiadiazolyl, furazanyl, pyridazinyl, triazinyl, cinnolinyl, benzimidazolyl, benzofuranyl, benzofurazanyl, benzothienyl, benzothiazolyl, benzoxazolyl, quinazolinyl, quinoxalinyl, naphthyridinyl, and furopyridinyl. Suitable heteroalicyclic groups in the compounds of the present invention contain one, two or three heteroatoms selected from N, O or S atoms and include, e.g., pyrrolidinyl, tetrahydrofuryl, dihydrofuryl, tetrahydrothienyl, tetrahydrothiopyranyl, piperidyl, morpholinyl, thiomorpholinyl, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, pyrazolidinyl, imidazolinyl, imidazolidinyl, 3-azabicyclo[3.1.0]hexyl, 3-azabicyclo[4.1.0]heptyl, 3H-indolyl, and quinolizinyl.

The groups above mentioned may be substituted at one or more available positions by one or more suitable groups such as OR′, ═O, SR′, SOR′, SO₂R′, OSO₂R′, OSO₃R′, NO₂, NHR′, N(R′)₂, ═N—R′, N(R′)COR′, N(COR′)₂, N(R′)SO₂R′, N(R′)C(═NR′)N(R′)R′, CN, halogen, COR′, COOR′, OCOR′, OCOOR′, OCONHR′, OCON(R′)₂, CONHR′, CON(R′)₂, CON(R′)OR′, CON(R′)SO₂R′, PO(OR′)₂, PO(OR′)R′, PO(OR′)(N(R′)R′), substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, wherein each of the R′ groups is independently selected from the group consisting of hydrogen, OH, NO₂, NH₂, SH, CN, halogen, COH, COalkyl, COOH, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list.

Suitable halogen groups or substituents in the compounds of the present invention include F, Cl, Br and I.

The term “pharmaceutically acceptable salts” refers to any salt which, upon administration to the patient is capable of providing (directly or indirectly) a compound as described herein. However, it will be appreciated that non-pharmaceutically acceptable salts also fall within the scope of the invention since those may be useful in the preparation of pharmaceutically acceptable salts. The preparation of salts can be carried out by methods known in the art.

For instance, pharmaceutically acceptable salts of compounds provided herein are synthesized from the parent compound, which contains a basic or acidic moiety, by conventional chemical methods. Generally, such salts are, for example, prepared by reacting the free acid or base forms of these compounds with a stoichiometric amount of the appropriate base or acid in water or in an organic solvent or in a mixture of both. Generally, nonaqueous media like ether, ethyl acetate, ethanol, 2-propanol or acetonitrile are preferred. Examples of the acid addition salts include mineral acid addition salts such as, for example, hydrochloride, hydrobromide, hydroiodide, sulfate, nitrate, phosphate, and organic acid addition salts such as, for example, acetate, trifluoroacetate, maleate, fumarate, citrate, oxalate, succinate, tartrate, malate, mandelate, methanesulfonate and p-toluenesulfonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts. Trifluoroacetate is one of the preferred pharmaceutically acceptable salt in the compounds of the invention.

The compounds of the invention may be in crystalline form either as free compounds or as solvates (e.g. hydrates, alcoholates, particularly methanolates) and it is intended that both forms are within the scope of the present invention. Methods of solvation are generally known within the art. The compounds of the invention may present different polymophic forms, it is intended that the invention encompasses all such forms.

Any compound that is a prodrug of a compound of formula I is within the scope of the invention. The term “prodrug” is used in its broadest sense and encompasses those derivatives that are converted in vivo to the compounds of the invention. Examples of prodrugs include, but are not limited to, derivatives and metabolites of the compounds of formula I that include biohydrolyzable moieties such as biohydrolyzable amides, biohydrolyzable esters, biohydrolyzable carbamates, biohydrolyzable carbonates, bio hydrolyzable ureides, and biohydrolyzable phosphate analogues. Preferably, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger “Medicinal Chemistry and Drug Discovery 6^{th} ed. (Donald J. Abraham ed., 2001, Wiley) and “Design and Applications of Prodrugs” (H. Bundgaard ed., 1985, Harwood Academic Publishers).

Any compound referred to herein is intended to represent such specific compound as well as certain variations or forms. In particular, compounds referred to herein may have asymmetric centres and therefore exist in different enantiomeric or diastereomeric forms. Thus any given compound referred to herein is intended to represent any one of a racemate, one or more enantiomeric forms, one or more diastereomeric forms, and mixtures thereof. Likewise, stereoisomerism or geometric isomerism about the double bond is also possible, therefore in some cases the molecule could exist as (E)-isomer or (Z)-isomer (trans and cis isomers). If the molecule contains several double bonds, each double bond will have its own stereoisomerism, that could be the same as, or different to, the stereoisomerism of the other double bonds of the molecule. Furthermore, compounds referred to herein may exist as atropisomers. All the stereoisomers including enantiomers, diastereoisomers, geometric isomers and atropisomers of the compounds referred to herein, and mixtures thereof, are considered within the scope of the present invention.

Furthermore, any compound referred to herein may exist as tautomers. Specifically, the term tautomer refers to one of two or more structural isomers of a compound that exist in equilibrium and are readily converted from one isomeric form to another. Common tautomeric pairs are amine-imine, amide-imidic acid, keto-enol, lactam-lactim, etc.

In addition, compounds referred to herein may exist in isotopically-labelled forms i.e. compounds which differ in the presence of one or more isotopically-enriched atoms. For example, compounds having the present structures except for the replacement of at least one hydrogen atom by deuterium or tritium, or the replacement of at least one carbon by ¹³C- or ¹⁴C-enriched carbon, or the replacement of at least one nitrogen atom by ¹⁵N-enriched nitrogen are within the scope of this invention.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

In compounds of general formula I, R₁ is preferably selected from substituted or unsubstituted C₁-C₁₂ alkyl and substituted or unsubstituted C₂-C₁₂ alkenyl, which may be branched or unbranched. More preferred alkyl and alkenyl groups, which may be branched or unbranched, are those having from 8 to about 12 carbon atoms; and even more preferably 9, 10 or 11 carbon atoms. It is particularly preferred that the alkyl and alkenyl groups are substituted by one or more suitable substituents, being the substituents preferably selected from OR′, ═O, SR′, SOR′, SO₂R′, SO₃R′, OSO₂R′, OSO₃R′, NO₂, NHR′, N(R′)₂, ═N—R′, N(R′)COR′, N(COR′)₂, N(R′)SO₂R′, N(R′)C(═NR′)N(R′)R′, CN, halogen, COR′, COOR′, OCOR′, OCOOR′, OCONHR′, OCON(R′)₂, CONHR′, CON(R′)₂, CON(R′)OR′, CON(R′)SO₂R′, PO(OR′)₂, PO(OR′)R′, PO(OR′)(N(R′)R′), substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, wherein each of the R′ groups is independently selected from the group consisting of hydrogen, OH, NO₂, NH₂, SH, CN, halogen, COH, COalkyl, COOH, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list. More preferably, substituents for the above mentioned alkyl and alkenyl groups are selected from OR′, OSO₂R′, OSO₃R′, halogen, OCOR′, OCOOR′, OCONHR′, OCON(R′)₂, CONHR′, and CON(R′)₂, wherein each of the R′ groups is independently selected from the group consisting of hydrogen, substituted or unsubstituted C₁-C₆ alkyl, substituted or unsubstituted C₂-C₆ alkenyl, substituted or unsubstituted C₂-C₆ alkynyl, substituted or unsubstituted aryl, and substituted or unsubstituted heterocyclic group; and even more preferred the substituent is OH. Most preferred R₁ is a substituted alkenyl group having 9, 10 or 11 carbon atoms; being 2-hydroxy-5,7-dimethyloct-3-enyl and 2-hydroxy-5-methyloct-3-enyl the most preferred.

Particularly preferred R₂, R₇, and R₁₁ are each independently selected from hydrogen and substituted or unsubstituted C₁-C₁₂ alkyl. More preferably R₂, R₇, and R₁₁ are each independently selected from hydrogen and substituted or unsubstituted C₁-C₆ alkyl. Even more preferably R₂, R₇, and R₁₁ are each independently selected from hydrogen, methyl, ethyl, n-propyl, iso-propyl and butyl, including n-butyl, tert-butyl, sec-butyl and iso-butyl; being hydrogen the most preferred group. Preferably R₂, R₇, and R₁₁ have the same meaning in the compounds of the invention.

Particularly preferred R₃ and R₄ are each independently selected from hydrogen, substituted or unsubstituted C₁-C₆ alkyl, CORₐ, and COORₐ, wherein Rₐ is selected from hydrogen and substituted or unsubstituted C₁-C₁₂ alkyl. Particularly preferred Rₐ is substituted or unsubstituted C₁-C₆ alkyl; and even more preferred is methyl, ethyl, n-propyl, iso-propyl and butyl, including n-butyl, tert-butyl, sec-butyl and iso-butyl. More preferably R₃ and R₄ are hydrogen. Preferably R₃ and R₄ have the same meaning in the compounds of the invention.

Particularly preferred R₅ and R₆ are each independently selected from hydrogen, substituted or unsubstituted C₁-C₁₂ alkyl, CORₐ, and COORₐ, wherein Rₐ is selected from hydrogen and substituted or unsubstituted C₁-C₁₂ alkyl. Particularly preferred Rₐ is substituted or unsubstituted C₁-C₆ alkyl; and even more preferred is methyl, ethyl, n-propyl, iso-propyl and butyl, including n-butyl, tert-butyl, sec-butyl and iso-butyl. More preferably R₅ and R₆ are hydrogen.

Particularly preferred R₈ and R₁₀ are each independently selected from hydrogen and halogen, wherein preferred halogen groups are Br and I. More preferably R₈ and R₁₀ are hydrogen.

Particularly preferred R₉ is OR_{c} wherein R_{c} is preferably selected from hydrogen, substituted or unsubstituted C₁-C₆ alkyl, CORₐ, and COORₐ, and wherein Rₐ is selected from hydrogen and substituted or unsubstituted C₁-C₁₂ alkyl. Particularly preferred Rₐ is substituted or unsubstituted C₁-C₆ alkyl; and even more preferred is methyl, ethyl, n-propyl, iso-propyl and butyl, including n-butyl, tert-butyl, sec-butyl and iso-butyl. More preferably R_{c} is hydrogen.

In additional preferred embodiments, the preferences described above for the different substituents are combined. The present invention is also directed to such combinations of preferred substitutions in the formula I above.

In the present description and definitions, when there are several groups Rₐ, R_{b}, or R_{c} present in the compounds of the invention, and unless it is stated explicitly so, it should be understood that they can be each independently different within the given definition, i.e. Rₐ does not represent necessarily the same group simultaneously in a given compound of the invention.

More particularly, the invention provides compounds of general formula II or pharmaceutically acceptable salts, tautomers, or prodrugs thereof

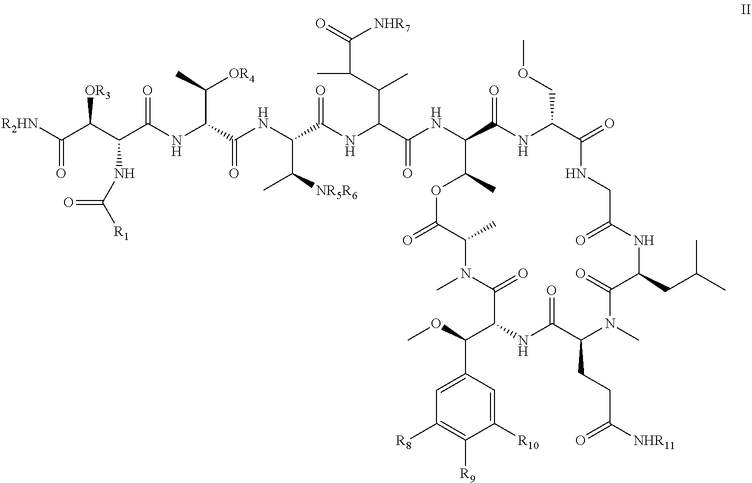

wherein R₁, R₂, R₃, R₄, R₅, R₆, R₇, R₈, R₉, R₁₀, and R₁₁ are as defined above.

Particularly preferred compounds of the invention are the following:

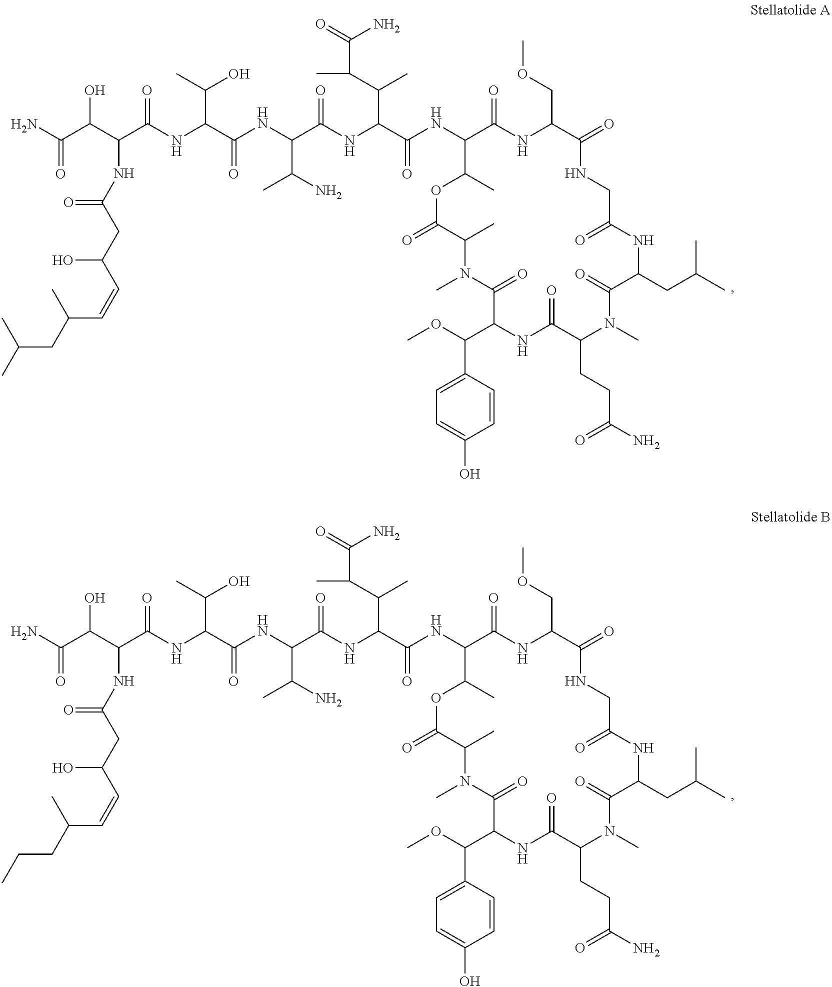

or pharmaceutically acceptable salts, tautomers, prodrugs or stereoisomers thereof.

Particularly preferred stereochemistry of Stellatolide A is

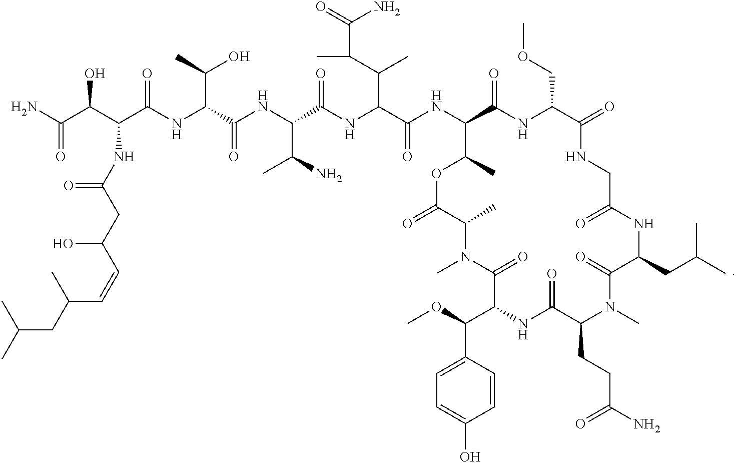

Particularly preferred stereochemistry of Stellatolide B is

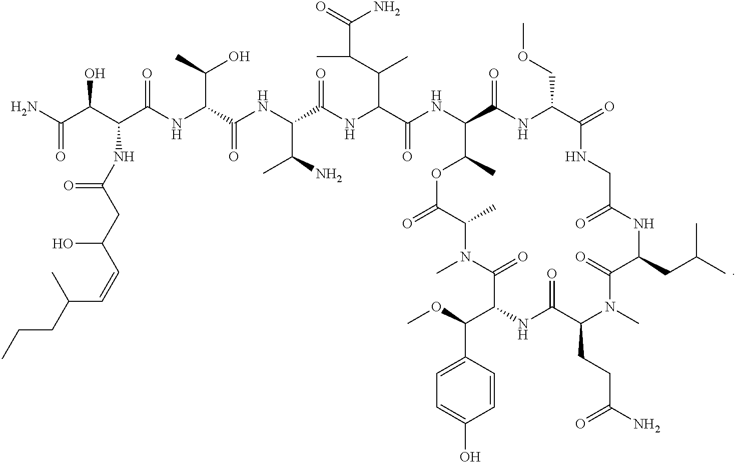

Stellatolides A and B were isolated from a sponge of the family Ancorinidae, genus Ecionemia, species Ecionemia acervus Bowerbank 1864. A sample of Ecionemia acervus Bowerbank 1864 was deposited in the Institute of Marine Sciences and Limnology of Universidad Nacional Autónoma of Mexico, with the reference code TLAR-477. This sponge was collected by hand using SCUBA diving in Tulear, Madagascar (23° 09.154′ S/43° 33.042′ E) at depths ranging between 12 and 29 m.

This sponge is a massive irregularly sponge, of approximately 5 cm thick in average and 6×2 cm in diameter, which is distributed in West Pacific, Indian Ocean, Indo-Pacific, Australia and New Zealand. When dried its color is brown and it is characterized by:
Megascleres: oxeas fusiform, abruptly pointed of 55-80 μm in size; ortothriaenes with rhabdome strongylate of 50-84 μm in size; and anatriaenes with rhabdome of 20-29 μm in size.
Microscleres: chiasters with terminal tylote actines of 8-15 μm in diameter, and microrhabde of 3 μm in size.
Skeletal arrangement: central condensation of oxeas from which spicular bundles arise radially toward the sponge surface. Trianes with the cladome placed immediately beneath the sponge surface and the rhabdome directed inwards.

Additionally, compounds of the invention can be obtained by modifying those already obtained from the natural source or by further modifying those already modified by using a variety of chemical reactions. Thus, hydroxyl groups can be acylated by standard coupling or acylation procedures, for instance by using acetic acid, acetyl chloride or acetic anhydride in pyridine or the like. Formate groups can be obtained by heating hydroxyl precursors in formic acid. Carbamates can be obtained by heating hydroxyl precursors with isocyanates. Hydroxyl groups can be converted into halogen groups through intermediate sulfonates for iodide, bromide or chloride, or directly using a sulfur trifluoride for fluorides; or they can be reduced to hydrogen by reduction of intermediate sulfonates. Hydroxyl groups can also be converted into alkoxy groups by alkylation using an alkyl bromide, iodide or sulfonate, or into amino lower alkoxy groups by using, for instance, a protected 2-bromoethylamine. Amido groups can be alkylated or acylated by standard alkylation or acylation procedures, for instance by using, respectively, KH and methyl iodide or acetyl chloride in pyridine or the like. Ester groups can be hydrolized to carboxylic acids or reduced to aldehyde or to alcohol. Carboxylic acids can be coupled with amines to provide amides by standard coupling or acylation procedures. When necessary, appropriate protecting groups can be used on the substituents to ensure that reactive groups are not affected. These protecting groups are well known for the skilled person in the art. A general review of protecting groups in organic chemistry is provided by Wuts, P. G. M. and Greene T. W. in Protecting groups in Organic Synthesis, 4^{th} Ed. Wiley-Interscience, and by Kocienski P. J. in Protecting Groups, 3^{rd} Ed. Georg Thieme Verlag. All these references are incorporated by reference in their entirety.

The procedures and reagents needed to prepare these derivatives are known to the skilled person and can be found in general textbooks such as March's Advanced Organic Chemistry 6th Edition 2007, Wiley Interscience.

An important feature of the above described compounds of formula I is their bioactivity and in particular their cytotoxic activity against tumor cells.

With this invention we provide pharmaceutical compositions of compounds of general formula I that possess cytotoxic activities and their use as anticancer agents. Thus the present invention further provides pharmaceutical compositions comprising a compound of this invention, or a pharmaceutically acceptable salt, tautomer, prodrug or stereoisomer thereof with a pharmaceutically acceptable carrier or diluent.

The term “carrier” refers to an adjuvant, excipient or vehicle with which the active ingredient is administered. Suitable pharmaceutical carriers are described in “Remington's Pharmaceutical Sciences” by E. W. Martin, 1995.

Examples of pharmaceutical compositions include any solid (tablets, pills, capsules, granules etc.) or liquid (solutions, suspensions or emulsions) composition for oral, topical or parenteral administration.

Administration of the compounds or compositions of the present invention may be by any suitable method, such as intravenous infusion, oral preparations, and intraperitoneal and intravenous administration. We prefer that infusion times of up to 24 hours are used, more preferably 1-12 hours, with 1-6 hours most preferred. Short infusion times which allow treatment to be carried out without an overnight stay in hospital are especially desirable. However, infusion may be 12 to 24 hours or even longer if required. Infusion may be carried out at suitable intervals of say 1 to 4 weeks. Pharmaceutical compositions containing compounds of the invention may be delivered by liposome or nanosphere encapsulation, in sustained release formulations or by other standard delivery means.

The correct dosage of the compounds will vary according to the particular formulation, the mode of application, and the particular situs, host and tumour being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

As used herein, the terms “treat”, “treating” and “treatment” include the eradication, removal, modification, or control of a tumor or primary, regional, or metastatic cancer cells or tissue and the minimization or delay of the spread of cancer.

The compounds of the invention have anticancer activity against several cancers types which include, but are not limited, lung cancer, colon cancer, and breast cancer.

Thus in alternative embodiments of the present invention, the pharmaceutical composition comprising the compounds of formula (I) as defined above is for the treatment of lung cancer, colon cancer or breast cancer.

### EXAMPLES

### Example 1

### Description of the Marine Organism and Collection Site

Ecionemia acervus Bowerbank 1864 was collected by hand using SCUBA diving in Tulear, Madagascar (23° 09.154′ S/43° 33.042′ E) at depths ranging between 12 and 29 m. The animal material was identified by Dr. José Luis Carballo (Universidad Nacional Autónoma of Mexico). A sample of the specimen was deposited in the Institute of Marine Sciences and Limnology of the Universidad Nacional Autónoma of Mexico, with the reference code TLAR-477.

### Example 2

### Isolation of Stellatolide A and B

The frozen specimen of Example 1 (176 g) was triturated and extracted with H₂O (3×250 mL) and then with a mixture of CH₃OH:CH₂Cl₂ (50:50, 3×250 mL) at 23° C. The aqueous layer was partitioned between Hexane, EtOAc and n-BuOH. Pure Stellatolide A (6.2 mg) in the form of its trifluoroacetate salt was obtained from the EtOAc extract by repeated semipreparative reversed phase HPLC: Atlantis dC₁₈, 10 μm, 10×150 mm, isocratic H₂O+0.1% TFA:CH₃CN+0.1% TFA 95:5 for 5 min, then gradient to 37% of CH₃CN+0.1% TFA in 1 min followed by isocratic H₂O+0.1% TFA:CH₃CN+0.1% TFA 63:37 in 22 min, UV detection, flow 3.8 mL/min to yield 6 fractions (H1 to H6); then fraction H2 (16.5 min of retention time) was injected into a XTerra MS C₁₈, 5 μm, 10×150 mm, isocratic H₂O+0.1% TFA:CH₃CN+0.1% TFA 65:35 in 14 min, UV detection, 3.8 mL/min to afford 3 fractions (H1 to H3); and finally fraction H1 (10.4 min of retention time) was injected into a XTerra Phenyl, 5 μm, 10×150 mm, isocratic H₂O+0.1% TFA:CH₃OH+0.1% TFA 40:60 in 11 min, UV detection, flow 3.5 mL/min. Stellatolide B (1.7 mg) in the form of its trifluoroacetate salt was isolated from a portion (850 mg) of the n-BuOH extract by preparative reversed phase HPLC (Atlantis dC₁₈, 5 μm, 19×150 mm, gradient H₂O+0.1% TFA:CH₃CN+0.1% TFA from 30 to 40% CH₃CN in 20 min, UV detection, flow 15.0 mL/min).

Stellatolide A: Amorphous colourless solid. (+)-HRMALDITOFMS m/z 1462.7764 [M+H]⁺ (calc. for C₆₆H₁₀₈N₁₅O₂₂, 1462.7787), 742.8856 [M+H+Na]²⁺ (calc. for C66H₁₀₈N₁₅O₂₂Na, 742.8840), 750.8687 [M+H+K]²⁺ (calc. for C₆₆H₁₀₈N₁₅O₂₂K, 750.8709), MS (ES) m/z 1463.6 [M+H]⁺. ¹H (500 MHz) and ¹³C NMR (125 MHz) see Table 1 (CD₃OD) and Table 2 (DMF-d₇).

Stellatolide B: Amorphous colourless solid. MS (ES) m/z 1449.5 [M+H]⁺, 1471.4 [M+Na]⁺. ¹H (500 MHz) and ¹³C NMR (125 MHz) see Table 3.

TABLE 1
¹H and ¹³C NMR data of Stellatolide A (CD₃OD)*

| | | |
|---|---|---|
| N^{o} | ¹³C | ¹H (Multiplicity, J) |

MeAla

| | | |
|---|---|---|
| 1 | 171.6 | — |
| 2 | 51.8 | 5.44 (q, 7.1) |
| 3 | 13.2 | 1.29 (d, 7.1) |
| NMe | 29.5 | 2.69 (s) |

βMeOTyr

| | | |
|---|---|---|
| 1 | 169.9 | 8.15 (d, 10.2) NH* |
| 2 | 53.7 | 4.93 (d, 9.5) |
| 3 | 84.3 | 4.55 (d, 9.5) |
| 4 | 129.4 | — |
| 5/5′ | 131.4 | 7.18 (d, 8.6) |
| 6/6′ | 116.0 | 6.80 (d, 8.6) |
| 7 | 158.7 | — |
| OMe | 56.9 | 3.11 (s) |

MeGln

| 1 | 170.7 | — | | |
|---|---|---|---|---|
| 2 | 55.6 | 4.79 (dd, 9.5, 6.6) | | |
| 3 | 25.0 | 1.64 (m) | | |
| | | 1.27 (m) | | |
| 4 | 32.0 | 1.68 (m) | | |
| 5 | 177.7 | | 7.78 (br s) | NH₂* |
| | | | 7.39 (br s) | |

| | | |
|---|---|---|
| NMe | 30.3 | 2.93 (s) |

Leu

| | | |
|---|---|---|
| 1 | 174.0 | 7.21 (d, 8.4) NH* |
| 2 | 49.2 | 4.73 (dd, 10.8, 3.5) |
| 3 | 40.5 | 1.67 (m) |
| | | 1.25 (m) |
| 4 | 26.1 | 1.67 (m) |
| 5 | 23.6^{b} | 0.96 (d, 6.6)^{c} |
| 4-Me | 21.4^{b} | 0.91 (d, 6.0)^{c} |

Gly

| | | |
|---|---|---|
| 1 | 172.2 | 9.08 (m) NH* |
| 2 | 44.1 | 3.96 (d, 17.1) |
| | | 3.51 (d, 17.1) |

MeOSer

| | | |
|---|---|---|
| 1 | 172.7 | 8.49 (d, 6.2) NH* |
| 2 | 55.2 | 4.51 (m) |
| 3 | 71.5 | 3.80 (dd, 10.0, 7.1) |
| | | 3.77 (dd, 10.0, 7.1) |
| OMe | 59.5 | 3.40 (s) |

Thr-1

| | | |
|---|---|---|
| 1 | 172.8 | 8.92 (d, 10.3) NH* |
| 2 | 57.3 | 5.21 (d, 3.1) |
| 3 | 71.6 | 5.60 (qd, 6.3, 3.1) |
| 4 | 14.8 | 1.19 (d, 6.3) |

DiMeGln

| 1 | 173.9 | Not observed | | |
|---|---|---|---|---|
| 2 | 59.0 | 4.11 (d, 9.2) | | |
| 3 | 37.0 | 2.46 (m) | | |
| 3-Me | 17.2 | 1.31 (d, 6.9) | | |
| 4 | 44.9 | 2.71 (m) | | |
| 4-Me | 14.0 | 1.33 (d, 7.2) | | |
| 5 | 182.1 | | 7.88 (br s)^{a} | NH₂* |
| | | | 7.10 (br s)^{a} | |

NH₂Thr

| | | |
|---|---|---|
| 1 | 171.2 | Not observed |
| 2 | 56.4 | 4.49 (m) |
| 3 | 49.1 | 3.97 (m) |
| 4 | 16.8 | 1.46 (d, 6.9) |
| NH₂ | — | Not observed |

Thr-2

| | | |
|---|---|---|
| 1 | 173.3 | 7.83 (d, 8.9) NH* |
| 2 | 60.0 | 4.35 (d, 2.4) |
| 3 | 67.4 | 4.48 (m) |
| 4 | 20.4 | 1.21 (d, 6.4) |

HOAsn

| 1 | 171.9 | 8.15 (d, 10.2) NH* | | |
|---|---|---|---|---|
| 2 | 59.1 | 4.75 (d, 4.2) | | |
| 3 | 72.5 | 4.61 (d, 4.2) | | |
| 4 | 176.5 | | 7.07 (br s)^{a} | NH₂* |
| | | | 6.80 (br s)^{a} | |

HDMN

| | | |
|---|---|---|
| 1 | 174.8 | — |
| 2 | 45.3 | 2.59 (dd, 13.8, 9.4) |
| | | 2.37 (dd, 13.7, 3.9) |
| 3 | 66.5 | 4.88 (m) |
| 4 | 130.9 | 5.37 (dd, 10.8, 9.0) |
| 5 | 139.4 | 5.22 (dd, 10.8, 10.8) |
| 6 | 31.3 | 2.64 (m) |
| 6-Me | 22.0 | 0.94 (d, 6.6) |
| 7 | 48.1 | 1.15 (m) |
| 8 | 26.9 | 1.57 (m) |
| 8-Me | 23.5^{d} | 0.90 (d, 6.5)^{e} |
| 9 | 23.0^{d} | 0.90 (d, 6.4)^{e} |

^{a}Assignments of NH₂ are interchangeable
^{b-e}Assignments can be interchangeable
HDMN: (Z)-3-hydroxy-6,8-dimethyl-4-nonenoyl
*NH and NH₂ chemical shifts and multiplicities were determined through experiments in CD₃OH

TABLE 2
¹H and ¹³C NMR data of Stellatolide A (DMF-d₇)

| | | |
|---|---|---|
| N^{o} | ¹³C | ¹H (Multiplicity, J) |

MeAla

| | | |
|---|---|---|
| 1 | 170.9 | — |
| 2 | 50.8 | 5.47 (q, 6.8) |
| 3 | 13.1 | 1.25 (d, 6.8) |
| NMe | 28.9 | 2.66 (s) |

βMeOTyr

| | | |
|---|---|---|
| 1 | 168.5 | 8.14 (d, 10.3) NH |
| 2 | 52.9 | 4.84 (dd, 10.3, 9.4) |
| 3 | 83.5 | 4.57 (d, 9.4) |
| 4 | 129.1 | — |
| 5/5′ | 131.6 | 7.22 (d, 8.4) |
| 6/6′ | 115.2 | 6.83 (d, 8.4) |
| 7 | 158.2 | — |
| OMe | 56.5 | 3.10 (s) |

MeGln

| | | |
|---|---|---|
| 1 | &^{b} | — |
| 2 | 55.2 | 4.70 (m) |
| 3 | 24.3 | 1.55 (m) |
| | | 1.28 (overlapped) |
| 4 | 31.2 | 1.67 (m) |
| 5 | 179.9 | — |
| NMe | 29.9 | 2.90 (under solvent) |

Leu

| | | |
|---|---|---|
| 1 | 173.2 | 7.02 (d, 9.3) NH |
| 2 | 48.3 | 4.71 (m) |
| 3 | 39.7 | 1.55 (m) |
| | | 1.31 (overlapped) |
| 4 | 26.1 | 1.55 (m) |
| 5 | 23.4^{c} | 0.85 (d, 6.6)^{d} |
| 4-Me | 22.5^{c} | 0.85 (d, 6.6)^{d} |

Gly

| | | |
|---|---|---|
| 1 | 170.2 | 9.19 (m) NH |
| 2 | 43.7 | 3.92 (dd, 16.7, 7.4) |
| | | 3.51 (dd 16.7, 5.2) |

MeOSer

| | | |
|---|---|---|
| 1 | 171.8 | 8.42 (d, 7.5) NH |
| 2 | 54.6 | 4.52 (m) |
| 3 | 70.9 | 3.85 (dd, 9.6, 8.8) |
| | | 3.61 (dd, 9.6, 6.6) |
| OMe | 58.9 | 3.37 (s) |

Thr-1

| | | |
|---|---|---|
| 1 | &^{b} | 9.24 (d, 10.4) NH |
| 2 | 56.4 | 5.24 (dd, 10.4, 2.0) |
| 3 | 70.8 | 5.61 (qd, 6.2, 2.0) |
| 4 | 14.8 | 1.19 (d, 6.2) |

DiMeGln

| | | |
|---|---|---|
| 1 | 173.1 | 10.31 (br s) NH |
| 2 | 58.5 | 4.27 (dd, 9.4, 3.8) |
| 3 | 36.5 | 2.50 (m) |
| 3-Me | 16.3 | 1.30 (d, 6.7) |
| 4 | 43.6 | 2.81 (dd, 6.7, 3.4) |
| 4-Me | 13.8 | 1.29 (d, 6.9) |

| | | | |
|---|---|---|---|
| 5 | &^{b} | 7.72 (br s)^{a} | NH₂ |
| | | 7.25 (br s)^{a} | |

NH₂Thr

| | | |
|---|---|---|
| 1 | 171.7 | 8.61 (d, 6.4) NH |
| 2 | 55.8 | 4.57 (m) |
| 3 | 49.0 | 3.93 (m) |
| 4 | 16.5 | 1.53 (d, 6.9) |
| NH₂ | — | 8.01 (under solvent) |

Thr-2

| | | |
|---|---|---|
| 1 | &^{b} | 7.71 (d, 8.5) NH |
| 2 | 59.1 | 4.32 (dd, 8.5, 2.1) |
| 3 | 66.6 | 4.42 (qd, 6.2, 2.1) |
| 4 | 20.0 | 1.14 (d, 6.5) |

HOAsn

| | | |
|---|---|---|
| 1 | 170.2 | 8.29 (d, 7.3) NH |
| 2 | 58.3 | 4.81 (m) |
| 3 | 72.4 | 4.65 (d, 3.6) |

| | | | |
|---|---|---|---|
| 4 | &^{b} | 7.86 (br s)^{a} | NH₂ |
| | | 7.52 (br s)^{a} | |

HDMN

| | | |
|---|---|---|
| 1 | 172.5 | — |
| 2 | 45.0 | 2.60 (dd 13.5, 9.2) |
| | | 2.35 (dd, 13.5, 4.0) |
| 3 | 66.8 | 4.84 (m) |
| 4 | 131.8 | 5.38 (dd, 10.8, 9.0) |
| 5 | 137.2 | 5.13 (dd, 10.8, 10.4) |
| 6 | 30.0 | 2.58 (m) |
| 6-Me | 21.8 | 0.90 (d, 6.7) |
| 7 | 47.4 | 1.10 (m) |
| 8 | 26.1 | 1.55 (m) |
| 8-Me | 23.2^{e} | 0.95 (d, 6.6)^{f} |
| 9 | 21.1^{e} | 0.88 (d, 6.7)^{f} |

^{a}Assignments of NH₂ are interchangeable
^{b}Carbonyl signals at these positions & (δ_{c}: 179.5, 175.6, 175.5, 170.4, and 169.0 ppm) were not assigned due to the failure of obtaining ¹H—¹³C long range connectivities
^{c-f}Assignments can be interchangeable

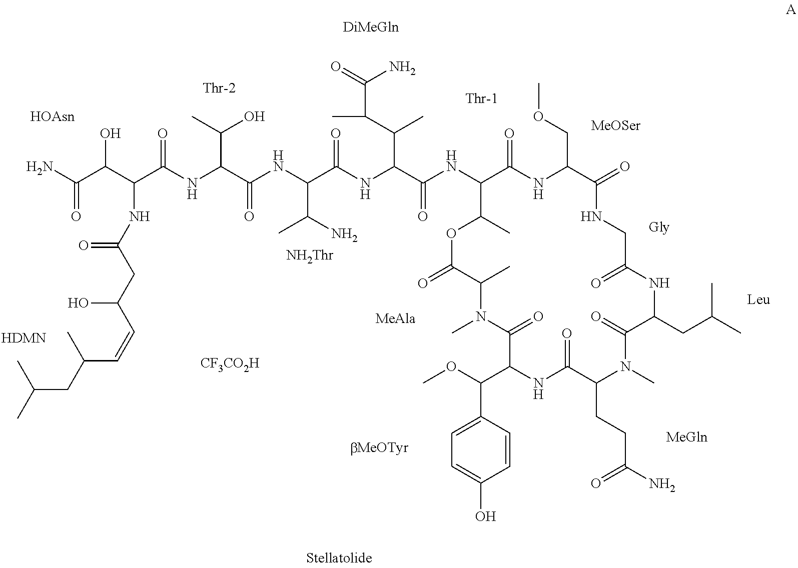

TABLE 3
¹H and ¹³C NMR data of Stellatolide B (CD₃OH)^{&}

| | | |
|---|---|---|
| N^{o} | ¹³C | ¹H (Multiplicity, J) |

MeAla

| | | |
|---|---|---|
| 1 | 170.1 | — |
| 2 | 51.0 | 5.43 (q, 6.7) |
| 3 | 11.8 | 1.28 (d, 6.7) |
| NMe | 28.2 | 2.68 (s) |

βMeOTyr

| | | |
|---|---|---|
| 1 | 168.4 | 8.15 (d, 9.2) NH^{d} |
| 2* | 52.6 | 4.93 (d, 9.3) |
| 3 | 83.2 | 4.54 (d, 9.3) |
| 4 | 128.0 | — |
| 5/5′ | 130.0 | 7.17 (d, 8.5) |
| 6/6′ | 114.7 | 6.79 (d, 8.5) |
| 7 | 157.2 | — |
| OMe | 55.6 | 3.10 (s) |

MeGln

| | | |
|---|---|---|
| 1 | 169.2 | — |
| 2* | 57.9 | 4.76 (m) |
| 3 | 23.9 | 1.57 (m) |
| | | 1.28 (m) |
| 4 | 30.9 | 1.69 (m) |
| 5 | 176.2 | 7.04 (brs)^{a}-6.81 (brs)^{a} |
| NMe | 29.0 | 2.91 (s) |

Leu

| | | |
|---|---|---|
| 1 | 172.2 | 7.21 (d, 9.2) NH |
| 2 | 48.3 | 4.75 (m) |
| 3 | 39.2 | 1.65 (m) |
| | | 1.29 (m) |
| 4 | 25.1 | 1.64 (m) |
| 5 | 22.2^{b} | 0.95 (d, 6.3)^{c} |
| 4-Me | 20.2^{b} | 0.89 (d, 6.3)^{c} |

Gly

| | | |
|---|---|---|
| 1 | 171.0 | 9.09 (dd, 7.1, 5.3) NH |
| 2 | 42.8 | 3.95 (dd, 17.2, 7.1) |
| | | 3.49 (dd, 17.2, 5.3) |

MeOSer

| | | |
|---|---|---|
| 1 | 171.0 | 8.51 (d, 7.5) NH |
| 2 | 54.3 | 4.48 (m) |
| 3 | 70.2 | 3.79 (dd, 10.0, 6.7) |
| | | 3.75 (dd, 10.0, 7.6) |
| OMe | 58.3 | 3.38 (s) |

Thr-1

| | | |
|---|---|---|
| 1 | 171.4 | 8.93 (d, 10.2) NH |
| 2 | 56.0 | 5.20 (dd, 10.2, 2.4) |
| 3 | 70.6 | 5.59 (qd, 6.5, 2.4) |
| 4 | 13.4 | 1.19 (d, 6.3) |

DiMeGln

| 1 | 172.3 | 9.95 (br s) NH | | |
|---|---|---|---|---|
| 2 | 57.9 | 4.09 (dd, 8.9, 3.0) | | |
| 3 | 35.8 | 2.45 (m) | | |
| 3-Me | 16.1 | 1.30 (d, 6.9) | | |
| 4 | 43.7 | 2.70 (m) | | |
| 4-Me | 12.8 | 1.32 (d, 7.2) | | |
| 5 | 180.9 | | 7.88 (br s)^{a} | NH₂ |
| | | | 7.10 (br s)^{a} | |

NH₂Thr

| | | |
|---|---|---|
| 1 | 169.3 | 8.73 (d, 6.6) NH |
| 2 | 55.3 | 4.47 (m) |
| 3 | 48.3 | 3.95 (m) |
| 4 | 15.6 | 1.45 (d, 6.8) |
| NH₂ | — | 7.69 (br s) |

Thr-2

| | | |
|---|---|---|
| 1 | 171.4 | 7.84 (d, 8.4) NH |
| 2 | 58.8 | 4.34 (dd, 8.4, 2.1) |
| 3 | 66.2 | 4.50 (m) |
| 4 | 19.3 | 1.20 (d, 6.3) |

HOAsn

| | | |
|---|---|---|
| 1 | 170.6 | 8.15 (d, 7.5) NH^{d} |
| 2* | 57.9 | 4.75 (d, 4.0) |
| 3 | 71.6 | 4.61 (d, 4.0) |

| | | | |
|---|---|---|---|
| 4 | 182.6 | 7.78 (br s)^{a} | NH₂ |
| | | 7.31 (br s)^{a} | |

HMN

| | | |
|---|---|---|
| 1 | 173.2 | — |
| 2 | 44.2 | 2.57 (dd, 13.9, 9.4) |
| | | 2.35 (dd, 13.9, 3.8) |
| 3* | 65.2 | 4.85 (under solvent) |
| 4 | 129.8 | 5.37 (dd, 10.9, 8.5) |
| 5 | 138.1 | 5.22 (dd, 10.9, 10.5) |
| 6 | 31.7 | 2.52 (m) |
| 6-Me | 20.6 | 0.94 (d, 6.6) |
| 7 | 39.7 | 1.19 (m) |
| 8 | 20.4 | 1.31 (m) |
| 9 | 13.3 | 0.89 (t, 7.1) |

^{a}Assignments of NH₂ are interchangeable
^{b-d}Assignments can be interchangeable
HMN: (Z)-3-hydroxy-6-methyl-4-nonenoyl
*¹H Chemical shifts and multiplicities for protons at these positions were obtained from an experiment measured in CD₃OD.
^{&}¹³C Chemical shifts determined through HSQC and HMBC experiments

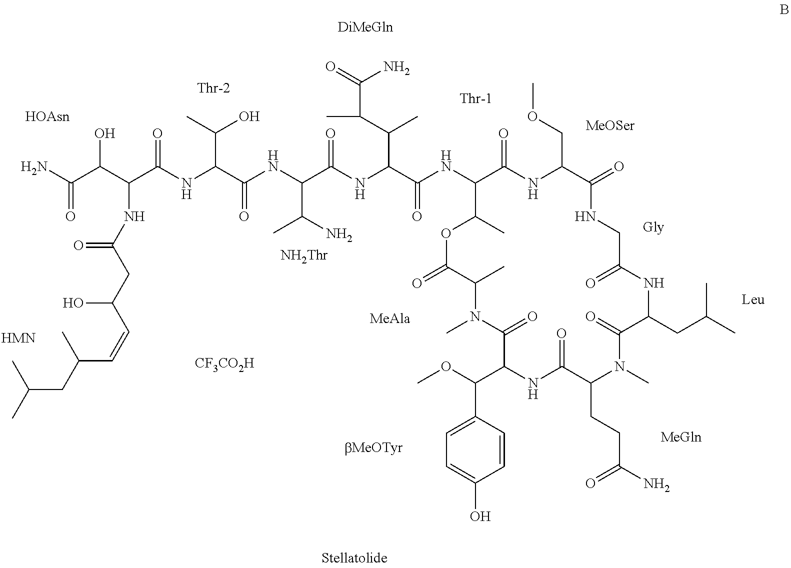

### Example 3

### Partial Determination of the Absolute Configuration of Aminoacid Residues in Stellatolides A and B

Marfey's analysis (P. Marfey, Carlsberg Res. Commun. 1984, 49, 591-596) was used to determine the absolute stereochemistry of the aminoacid residues in both, stellatolide A and B.

0.2 mg of Stellatolide A were dissolved in 0.5 mL of 6N HCl in a sealed vial and heated at 110° C. for 16 h. The solvent was evaporated under a N₂ stream, the residue was dissolved in 50 μL of water, and 0.5 mg of fluorodinitrophenyl-5-L-alaninamide (L-FDAA, Marfey's reagent) in 100 μL of acetone and 40 μL of 1N aqueous NaHCO₃ were added. The resulting mixture was heated at 40° C. for 1 h and, after cooling to room temperature, neutralised with 100 μL of 2N HCl. Finally, the mixture was filtrated (45 μm filter) and diluted with 800 μL of water prior to HPLC-MS analysis.

Standards of all possible stereoisomers of the aminoacid residues present in Stellatolide A, except 2,3-dimethylglutamine (DiMeGln), were derivatized in the same manner as the peptide hydrolysate. Relative retention times to unreacted L-FDAA of both, the derivatized hydrolysate and the derivatized aminoacid standards, were determined by reversed phase HPLC-MS: Symmetry C18, 5 μm, 4.6×150 mm, gradient H₂O+0.04% TFA:CH₃CN+0.04% TFA from 20% to 50% CH₃CN+0.04% TFA in 30 min, UV (215 and 254 nm) and MS/ESI+ detection, 0.8 mL/min.

Comparison of these retention times unambiguously confirmed the presence in Stellatolide A of L-Leu (S), D-MeOSer (R), D-allo-Thr (2R,3R) (both residues), (2S,3S)—NH₂Thr and (2R,3S)—OHAsn. β-methoxytyrosine (βMeOTyr) underwent degradation under acidic hydrolysis conditions, and therefore an alternative way was used to determine its absolute stereochemistry. The aminoacid residue was first converted to β-methoxyaspartic acid by ozonolysis and oxidative work-up, and then the modified peptide was subjected to acid hydrolysis, derivatization, and HPLC-MS analysis. To do that, a stream of ozone in O₂ was bubbled through a cooled solution of Stellatolide A (0.4 mg) in MeOH (0.5 mL) at −78° C. for 1 h. Hydrogen peroxide (35%, 4 drops) was added and the reaction mixture was allowed to stand at room temperature overnight. The solvent was removed under a stream of N₂ and the resultant residue was hydrolyzed in 0.5 mL of 6N HCl heating at 160° C. for 16 h in a sealed vial; after cooling to room temperature the mixture was dried under a N₂ stream and immediately subjected to a modified Marfey's derivatization procedure (A. Zampella et al. Org. Lett., 7(16), 3585-3588): the residue was dissolved in 80 μL of a 2:3 solution of Et₃N:MeCN and 75 μg of L-FDAA in 75 μL of MeCN:acetone (1:2) were added; the mixture was heated at 70° C. for 1 h, cooled to room temperature, neutralized with 50 μL of 2N HCl and dried with N₂. The residue was dissolved in 1 mL of MeCN:H₂O 1:1 and filtrated (0.45 μm) for HPLC-MS analysis. All four of the aminoacid standards were ozonized and derivatized following a similar procedure. Relative retention times to unreacted L-FDAA, obtained by HPLC-MS performed in the previously described conditions, confirmed the residue in Stellatolide A to be (2R,3R)-βMeOTyr.

Due to the overlapping of the retention times for the L-FDAA derivatives of the pairs D and L N-methylglutamine (MeGln), and D and L N-methylalanine (MeAla), different chromatographic conditions were employed for the determination of the absolute stereochemistry of these two aminoacids.

Injection on a Symmetry C18 column (5 μm, 4.6×150 mm, isocratic H₂O+0.04% TFA:CH₃CN+0.04% TFA 90:10 for 20 min followed by a gradient to 27% CH₃CN+0.04% TFA in 45 min, UV (215 and 254 nm) and MS/ESI+ detection, 0.8 mL/min) allowed to determine the presence of L-MeGln in Stellatolide A.

The L absolute configuration of N-methylalanine (MeAla), was established using the following chromatographic conditions: Symmetry C18, 5 μm, 4.6×150 mm, gradient H₂O+0.1% TFA : CH₃OH+0.1% TFA from 45% to 50% CH₃OH+0.1% TFA in 15 min, UV (215 and 254 nm) and MS/ESI+ detection, 0.8 mL/min.

Following similar procedures to those described for Stellatolide A, the presence of L-Leu (S), D-MeOSer (R), D-allo-Thr (2R,3R) (both residues), (2S,3S)—NH₂Thr, (2R,3S)—OHAsn, (2R,3R)-βMeOTyr, L-MeGln, and L-MeAla was unambiguously confirmed in Stellatolide B.

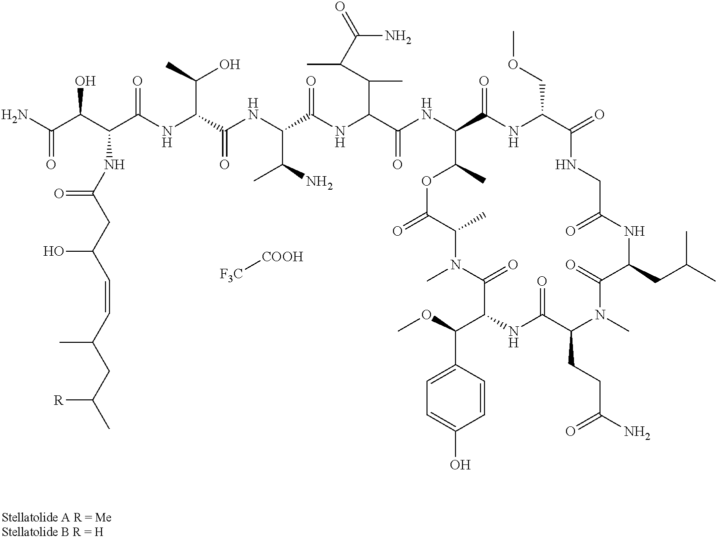

### Example 4

### Bioassays for the Detection of Antitumor Activity

The aim of this assay is to evaluate the in vitro cytostatic (ability to delay or arrest tumor cell growth) or cytotoxic (ability to kill tumor cells) activity of the samples being tested.

### Cell Lines

| Name | N^{o} ATCC | Species | Tissue | Characteristics |
|---|---|---|---|---|
| A549 | CCL-185 | human | lung | lung carcinoma (NSCLC) |
| HT29 | HTB-38 | human | colon | colorectal adenocarcinoma |
| MDA-MB- | HTB-26 | human | breast | breast adenocarcinoma |
| 231 | | | | |

### Evaluation of Cytotoxic Activity Using the SBR Colorimetric Assay

A colorimetric assay, using sulforhodamine B (SRB) reaction has been adapted to provide a quantitative measurement of cell growth and viability (following the technique described by Skehan et al. J. Natl. Cancer Inst. 1990, 82, 1107-1112).

This form of assay employs SBS-standard 96-well cell culture microplates (Faircloth et al. Methods in Cell Science, 1988, 11(4), 201-205; Mosmann et al, Journal of Immunological Methods, 1983, 65(1-2), 55-63). All the cell lines used in this study were obtained from the American Type Culture Collection (ATCC) and derive from different types of human cancer.

Cells were maintained in Dulbecco's Modified Eagle Medium (DMEM) supplemented with 10% Fetal Bovine Serum (FBS), 2 mM L-glutamine, 100 U/mL penicillin and 100 U/mL streptomycin at 37° C., 5% CO₂ and 98% humidity. For the experiments, cells were harvested from subconfluent cultures using trypsinization and resuspended in fresh medium before counting and plating.

Cells were seeded in 96 well microtiter plates, at 5×10³-7.5×10³ cells per well in aliquots of 150 μL, and allowed to attach to the plate surface for 18 hours (overnight) in drug free medium. After that, one control (untreated) plate of each cell line was fixed (as described below) and used for time zero reference value. Culture plates were then treated with test compounds (50 μL aliquots of 4× stock solutions in complete culture medium plus 4% DMSO) using ten serial dilutions (concentrations ranging from 10 to 0.00262 μg/mL) and triplicate cultures (1% final concentration of DMSO). After 72 hours treatment, the antitumor effect was measured by using the SRB methodology: Briefly, cells were washed twice with PBS, fixed for 15 min in 1% glutaraldehyde solution at room temperature, rinsed twice in PBS, and stained in 0.4% SRB solution for 30 min at room temperature. Cells were then rinsed several times with 1% acetic acid solution and air-dried at room temperature. SRB was then extracted in 10 mM trizma base solution and the absorbance measured in an automated spectrophotometric plate reader at 490 nm. Effects on cell growth and survival were estimated by applying the NCI algorithm (Boyd M R and Paull K D. Drug Dev. Res. 1995, 34, 91-104).

Using the mean±SD of triplicate cultures, a dose-response curve was automatically generated using nonlinear regression analysis. Three reference parameters were calculated (NCI algorithm) by automatic interpolation: GI₅₀=compound concentration that produces 50% cell growth inhibition, as compared to control cultures; TGI=compound concentration that produces total cell growth inhibition (cytostatic effect), as compared to control cultures, and LC₅₀=compound concentration that produces 50% net cell killing (cytotoxic effect).

Table 4 illustrates data on the biological activity of compounds of the present invention.

TABLE 4
Cytotoxicity assay-Activity Data (Molar) of Stellatolide A and B.

| | | Stellatolide A | Stellatolide B |
|---|---|---|---|
| MDA-MB-231 | GI₅₀ | 3.30E−07 | 7.04E−07 |
| | TGI | 4.95E−07 | 1.09E−06 |
| | LC₅₀ | 7.61E−07 | 1.73E−06 |
| HT29 | GI₅₀ | 6.03E−07 | 1.15E−06 |
| | TGI | 8.88E−07 | 1.60E−06 |
| | LC₅₀ | 1.33E−06 | 2.37E−06 |
| A549 | GI₅₀ | 1.01E−07 | 6.40E−07 |
| | TGI | 1.97E−07 | 1.09E−06 |
| | LC₅₀ | 4.06E−07 | 1.92E−06 |

1. A compound of general formula I 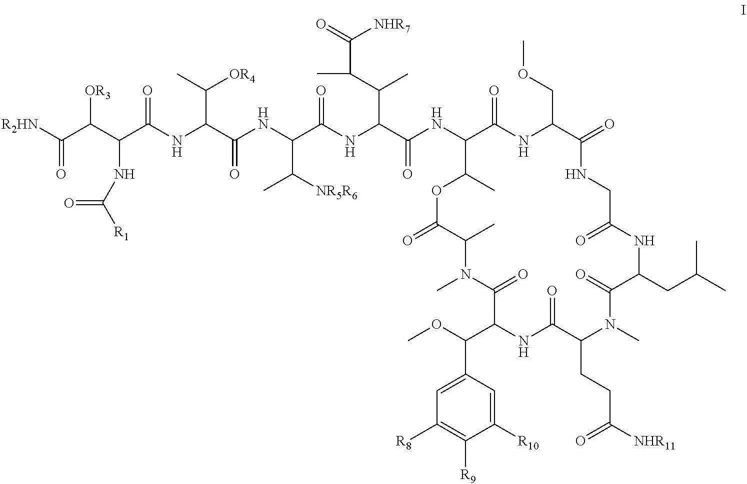
wherein
R₁ is selected from substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group;
each R₂, R₇, and R₁₁ is independently selected from hydrogen, CORₐ, COORₐ, CONRₐR_{b}, substituted or unsubstituted C₁-C₁₂ alkyl, substituted or unsubstituted C₂-C₁₂ alkenyl, and substituted or unsubstituted C₂-C₁₂ alkynyl;
each R₃ and R₄ is independently selected from hydrogen, CORₐ, COORₐ, CONRₐR_{b}, SO₂Rₐ, SO₃Rₐ, substituted or unsubstituted C₁-C₁₂ alkyl, substituted or unsubstituted C₂-C₁₂ alkenyl, and substituted or unsubstituted C₂-C₁₂ alkynyl;
each R₅ and R₆ is independently selected from hydrogen, CORₐ, COORₐ, substituted or unsubstituted C₁-C₁₂ alkyl, substituted or unsubstituted C₂-C₁₂ alkenyl, and substituted or unsubstituted C₂-C₁₂ alkynyl;
each R₈, R₉, and R₁₀ is independently selected from hydrogen, OR_{c}, CORₐ, COORₐ, CONRₐR_{b}, CN, NRₐR_{b}, halogen, substituted or unsubstituted C₁-C₁₇ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group;
R_{c} is selected from hydrogen, CORₐ, COORₐ, CONRₐR_{b}, SO₂Rₐ, SO₃Rₐ, substituted or unsubstituted C₁-C₁₂ alkyl, substituted or unsubstituted C₂-C₁₂ alkenyl, and substituted or unsubstituted C₂-C₁₂ alkynyl; and
each Rₐ and R_{b} is independently selected from hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group;
or a pharmaceutically acceptable salt, tautomer, prodrug or stereoisomer thereof.

2. A compound according to claim 1, wherein R₁ is selected from substituted or unsubstituted C₁-C₁₂ alkyl and substituted or unsubstituted C₂-C₁₂ alkenyl, which may be branched or unbranched.

3. A compound according to claim 1, wherein R₁ is selected from substituted C₈-C₁₂ alkyl and substituted C₈-C₁₂ alkenyl, wherein they are independently substituted by one or more substituents selected from OR′, OSO₂R′, OSO₃R′, halogen, OCOR′, OCOOR′, OCONHR′, OCON(R′)₂, CONHR′, and CON(R′)₂, wherein each of the R′ groups is independently selected from the group consisting of hydrogen, substituted or unsubstituted C₁-C₆ alkyl, substituted or unsubstituted C₂-C₆ alkenyl, substituted or unsubstituted C₂-C₆ alkynyl, substituted or unsubstituted aryl, and substituted or unsubstituted heterocyclic group.

4. A compound according to claim 1, wherein R₁ is selected from 2-hydroxy-5,7-dimethyloct-3-enyl and 2-hydroxy-5-methyloct-3-enyl.

5. A compound according to claim 1, wherein R₂, R₇, and R₁₁ are each independently selected from hydrogen and substituted or unsubstituted C₁-C₆ alkyl.

6. A compound according to claim 5, wherein R₂, R₇, and R₁₁ are hydrogen.

7. A compound according to claim 1, wherein R₃ and R₄ are each independently selected from hydrogen, substituted or unsubstituted C₁-C₆ alkyl, CORₐ, and COORₐ, wherein Rₐ is selected from hydrogen and substituted or unsubstituted C₁-C₁₂ alkyl.

8. A compound according to claim 7, wherein R₃ and R₄ are hydrogen.

9. A compound according to claim 1, wherein R₅ and R₆ are each independently selected from hydrogen, substituted or unsubstituted C₁-C₁₂ alkyl, CORₐ, and COORₐ, wherein Rₐ is selected from hydrogen and substituted or unsubstituted C₁-C₁₂ alkyl.

10. A compound according to claim 9, wherein R₅ and R₆ are hydrogen.

11. A compound according to claim 1, wherein R₈ and R₁₀ are each independently selected from hydrogen and halogen.

12. A compound according to claim 11, wherein R₈ and R₁₀ are hydrogen.

13. A compound according to claim 1, wherein R₉ is OR_{c}, wherein R_{c} is selected from hydrogen, substituted or unsubstituted C₁-C₆ alkyl, CORₐ, and COORₐ, and wherein Rₐ is selected from hydrogen and substituted or unsubstituted C₁-C₁₂ alkyl.

14. A compound according to claim 13, wherein R_{c} is hydrogen.

15. A compound according to claim 1, having the following structure: 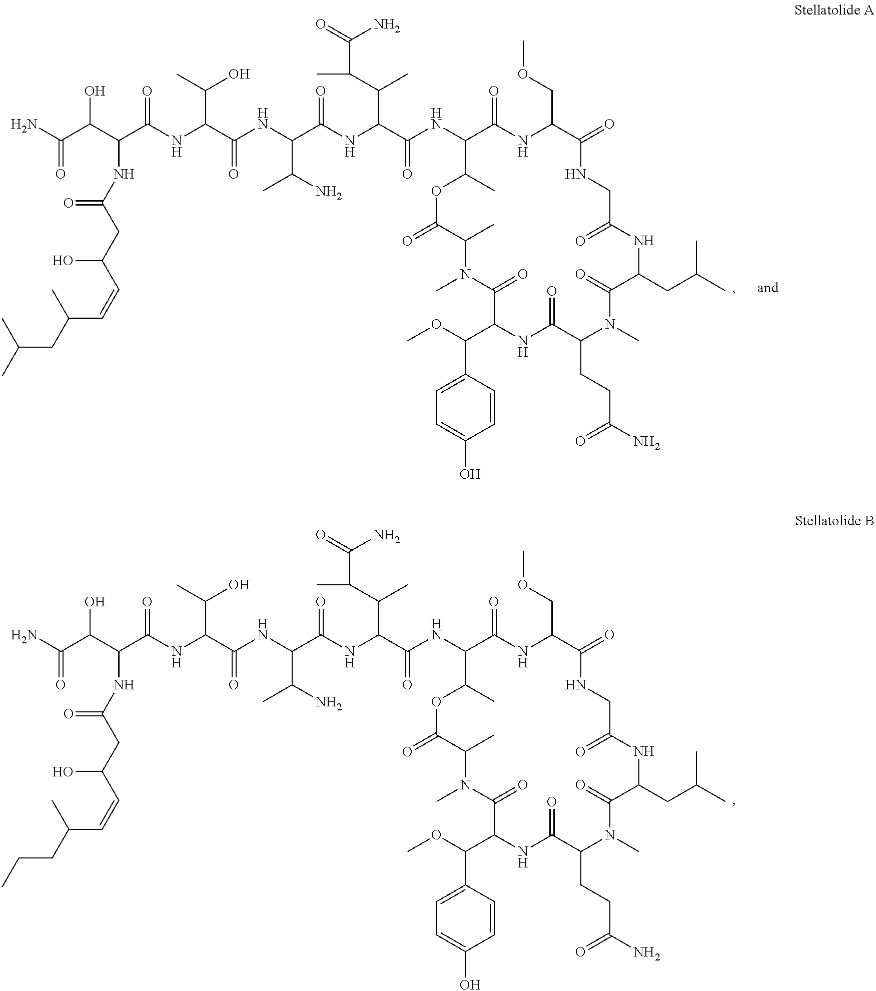
or a pharmaceutically acceptable salt, tautomer, prodrug or stereoisomer thereof.

16. A pharmaceutical composition comprising a compound according to any preceding claim, or a pharmaceutically acceptable salt, tautomer, prodrug or stereoisomer thereof, and a pharmaceutically acceptable carrier or diluent.

17. (canceled)

18. (canceled)

19. A method of treating a patient affected by cancer which comprises administering to said affected individual in need thereof a therapeutically effective amount of a compound as defined in any of claims 1 to 15.
